(19) United States
(12) Patent Application Publication
(10) Pub. No.: US 2016/0151134 A1
(43) Pub. Date: Jun. 2, 2016

(54) FORMULATIONS FOR CONTROL AND PREVENTION OF PERIODONTAL AND PERIMPLANT DISEASES AND OTHER DISEASES AND CONDITIONS OF THE MOUTH, AND SUPRA AND SUB-GINGIVAL DELIVERY METHODS AND SYSTEMS FOR SUCH FORMULATIONS

(71) Applicants: George N. Britt, Birmingham, AL (US); William E. Fixler, Birmingham, AL (US)

(72) Inventors: George N. Britt, Birmingham, AL (US); William E. Fixler, Birmingham, AL (US)

(21) Appl. No.: 15/018,642

(22) Filed: Feb. 8, 2016

Related U.S. Application Data
(63) Continuation of application No. PCT/US2014/050351, filed on Aug. 8, 2014.
(60) Provisional application No. 61/864,362, filed on Aug. 9, 2013.
(60) Provisional application No. 61/864,390, filed on Aug. 9, 2013.

(51) Int. Cl. A61C 17/02; A61K 9/00; A61C 19/06; A61K 31/65

(52) U.S. Cl. CPC A61C 17/0202; A61K 31/65; A61K 9/0063; A61C 19/063

(57) ABSTRACT

A chemical formulation that is adapted to be used for the control and prevention of periodontal and perimplant conditions and diseases. The formulation comprises a chemical agent, such as an antibiotic. Also included are topical anesthetics. Delivery systems, kits used for delivery, and delivery methods are also contemplated.

### FIELD

This disclosure relates to the control and prevention of periodontal and perimplant diseases.

### BACKGROUND

Periodontal and perimplant diseases can be painful and costly. Avoiding or minimizing such diseases helps patients, dental professionals, insurance companies and employers.

### SUMMARY

Featured in this disclosure are chemical formulations that are adapted to be used for the control and prevention of periodontal and perimplant diseases and other diseases and conditions of the mouth. Also featured herein are delivery systems that are adapted to be used by dental and veterinary professionals to deliver these formulations supra or sub-gingivally. The formulations and delivery systems and kits apply equally to use on humans and other mammals, for example veterinary usage, non-limiting examples of which include usage on pets and domestic mammals.

Also featured in this disclosure are methods for the supra and subgingival delivery of chemical formulations that are adapted to be used for the control and prevention of periodontal and perimplant diseases and other diseases and conditions of the mouth. The methods are adapted to be practiced by dental and veterinary professionals to deliver any and all types of agents supra or sub-gingivally. The methods apply equally to use on humans and veterinary usage, non-limiting examples of which include usage on pets and domestic/domesticated mammals.

The formulations include but are not limited to various chemical agents such as antimicrobials, antibiotics and topical anesthetics. One or more of the formulations can be antimicrobial, antibiotic and/or anti-collagenase, and can bind to calcium so that they remain in the gingival pocket for an extended period and thus are slowly released over a period of time to be clinically effective for an extended period. The formulations can have an effective shelf life of five months or more, without refrigeration.

The antibiotics are preferably one or more members of the tetracycline antibiotics, including for example tetracycline, doxycycline and/or minocycline. The topical anesthetics are preferably one or more of lidocaine and prilocaine. The chemical agent(s) are carried in a medium that can be delivered supra or sub-gingivally. The medium can be, for example, a liquid, a paste or a gel.

Specific, non-limiting examples of the subject formulations include the following:
A formulation particularly adapted for use in periodontal surgery comprising 100 mg/ml antibiotic (hereinafter sometimes referred to as “surgical formulation”)
A formulation particularly adapted for use in endodontic treatments comprising 50 mg/ml antibiotic (hereinafter sometimes referred to as “endodontic formulation”)
A formulation particularly adapted for use in periodontal maintenance comprising 50 mg/ml antibiotic (hereinafter sometimes referred to as “maintenance formulation”)
A formulation particularly adapted for use as a topical anesthetic that is administered before, during or after various dental procedures including but not limited to a periodontal, preventive, implant, surgical, restorative and endodontic procedures comprising 2.5% prilocaine and 2.5% lidocaine (hereinafter sometimes referred to as “anesthetic formulation”)

The subject formulations are typically but not necessarily suspensions of the antibiotic or anesthetic agents in an appropriate suspension and delivery vehicle such as a liquid, paste or gel, that is able to be delivered via a syringe or other delivery device with a delivery tip. The antibiotic and anesthetic formulations can be made by wetting the antibiotic powder or powdered anesthetic agents with a suspension medium, and then serially diluting the formulation with the suspension medium, to the final concentration. A homogenizer is used to homogenize the formulation. This can be done with a mortar and pestle. If a tetracycline HCl is used then the pH may need to be adjusted, preferably into the range of from about 3.5 to about 9. This can be done with sodium hydroxide or another basic substance.

In one aspect this disclosure features a chemical formulation that is adapted to be used for the control and prevention of periodontal and perimplant conditions and diseases, the formulation comprising a chemical agent such as an antibiotic. An antibiotic may comprise one or more members of the tetracycline antibiotics, including, for example tetracycline, doxycycline and/or minocycline. The topical anesthetics may include lidocaine and/or prilocaine. The chemical agent(s) may be carried in a medium that can be delivered supra or sub-gingivally, wherein the medium is a liquid, a paste or a gel.

Also featured is a delivery system that may be adapted to be used by dental or veterinary professionals to deliver any of the subject formulations, supra-gingivally and/or sub-gingivally. The delivery system may comprise a pre-filled syringe or other delivery device. The delivery system may further include one or more delivery tips that can be coupled to a syringe or other delivery device. The tips may be straight, bent or curved, for example.

Further featured is a kit which includes a pre-filled syringe or other delivery device as described above, and one or more tips as described above, packaged together in a kit for use by a dental or veterinary professional.

Still further featured herein is a method for supra and/or subgingival delivery of any of the above formulations, comprising attaching a delivery tip to a pre-filled syringe or other delivery device, and delivering the agent supragingivally or into a subgingival pocket via the tip. A bristled tip may be used, where the wetted bristles are used to push the agent interproximally, supragingivally, or subgingivally into the pocket. The bristles may be long enough and flexible enough to be able to penetrate into shallow pockets. A tip with an elongated distal delivery tube may be used to deliver the agent into deeper pockets and other areas such as furcations and implant components. The delivery tube may be long enough to be able to penetrate into deeper pockets. The tip may be able to be bent, so as to reach different areas of the mouth.

Still further featured is a method for supra or sub-gingival delivery of a chemical agent or formulation into the mouth of a mammal, comprising attaching a delivery tip to a pre-filled syringe or other delivery device, and delivering the agent or formulation into the mouth via the tip. A bristled tip may be used, where the wetted bristles are used to push the agent or formulation interproximally, supragingivally, or subgingivally into a pocket. The bristles may be long enough and flexible enough to be able to penetrate into shallow pockets. A tip with an elongated distal delivery tube may be used to deliver the agent or formulation. The delivery tube may be long enough to be able to penetrate into deeper subgingival pockets and other areas such as furcations and implant components. The tip may be able to be bent, so as to reach different areas of the mouth. The tip may be able to be cut or otherwise shortened if desired.

### BRIEF DESCRIPTION OF THE DRAWINGS

FIG. 1 is a perspective view of a delivery tip.

FIG. 2 is a cross-sectional view of a delivery tip.

FIG. 3 illustrates an assembled delivery kit.

FIG. 4 illustrates a delivery tip.

### DETAILED DISCLOSURE

One non-limiting example of an appropriate compounding suspension medium for the antibiotic formulations is Ora-Blend™ from Intercare, Melbourne Australia. Ora-Blend is available with sugar or as a sugar-free medium, called “Ora-Blend SF,” which is an aqueous-based, sweetened vehicle consisting of a synergistic blend of suspending agents that have a high degree of colloidal activity. The suspending agents form a structured, gel-like matrix which suspend particles and allow for little settling. Ora-Blend SF is buffered to a slightly acidic pH to help reduce degradation of medicinal agents through oxidation. An anti-foam agent is incorporated into Ora-Blend SF to allow for vigorous shaking with minimal foam. Its ingredients are: purified water, sorbitol, glycerin, flavoring, microcrystalline cellulose, carboxymethylcellulose sodium, xanthan gum, carrageenan, calcium sulfate, trisodium phosphate and sodium saccharin, sodium phosphate, citric acid and sodium citrate as buffers, and dimethicone antifoam emulsion. It is preserved with methylparaben, propylparaben and potassium sorbate. Its specification are: Appearance: Opaque, pinkish liquid with a pH of approximately 4.2; Taste: Sweet citrus-berry flavour; Viscosity: Approximately 1000 cps at 25° C. via Brookfield viscometer; Osmolality: 1073 mOsm/Kg.

One non-limiting example of an appropriate compounding suspension medium for the anesthetic formulation is an appropriate vehicle including but not limited to a biphasic reversible copolymers such as #188 and #407 as in Oraqix® available from DENTSPLY Pharmaceutical of York, Pa., USA. The anesthetic can be delivered in the same or similar manners as described herein for the antibiotic formulations.

Other suspension media are contemplated herein. One example could be water with an increased viscosity through use of a thickener such as carrageenan, or carbomer 940 NF, or the like.

The delivery systems that are adapted to be used by dental and/or veterinary professionals to deliver the antibiotic formulations supra or sub-gingivally comprise pre-filled syringes or other delivery devices that include an effective amount of a formulation for the specific procedure being conducted. The delivery systems also include one or more delivery tips that are either already coupled to the output of the syringe, or are adapted to be coupled to the output of the syringe just before delivery of the syringe contents. The delivery tips are adapted to deliver formulations supragingivally or into the open top of or directly into the volume of gingival, perimplant or periodontal pockets. The formulations delivered in this manner will fully or partially fill the pocket and this will bathe the root of the tooth, the surface of the implant and/or the alveolar bone in the recipient.

This disclosure also features pre-packaged kits that are adapted to be used by dental or veterinary professionals to deliver the antibiotic and anesthetic formulations supra or sub-gingivally. Each kit includes a pre-filled syringe that includes an effective amount of a formulation for the specific procedure being conducted. The kit also includes one or more delivery tips that are either already coupled to the output of the syringe, or are adapted to be coupled to the output of the syringe just before delivery of the syringe contents. The delivery tips are adapted to deliver formulations supragingivally or into the open top of or directly into the volume of gingival, perimplant or periodontal pockets.

In one non-limiting embodiment the pre-filled syringes are configured as follows:
The surgical formulation has 2 ml of the correct antibiotic formulation
The endodontic formulation has 0.5 ml of the correct antibiotic formulation
The full mouth maintenance formulation has 2 ml of the correct antibiotic formulation
The partial mouth maintenance formulation has 0.5 ml of the correct antibiotic formulation
The anesthetic formulation has 2 ml of the correct anesthetic formulation

The disclosure is not limited to pre-packaged kits. The formulations could be dispensed in other manners as is known in the field. One example is that the formulations could be dispensed out of multi-dose containers.

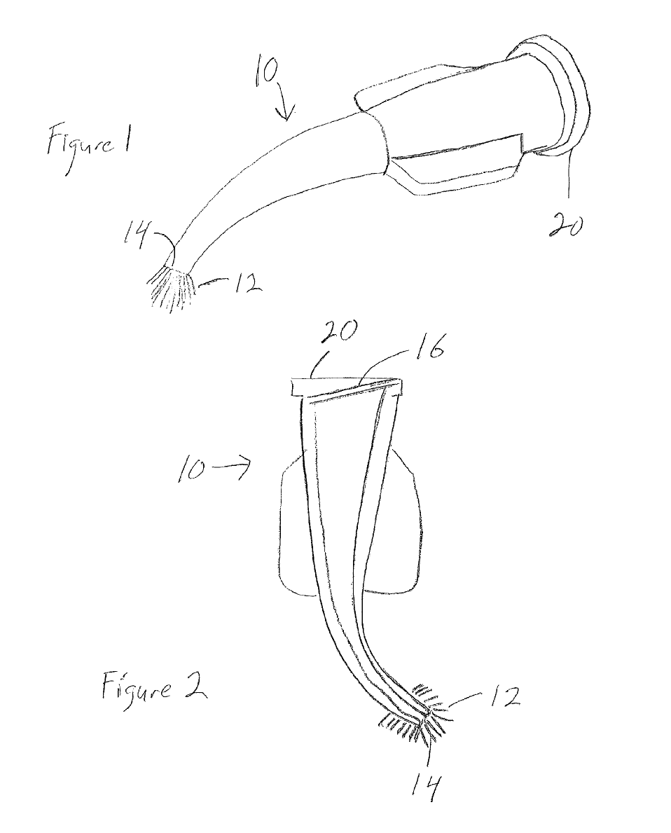

The delivery tips can include one or more delivery tips, including but not limited to the tips disclosed herein, which are exemplary of tips but are not limiting of the tips that can be used. Two examples of tips are illustrated. One tip 10, FIGS. 1 and 2, is a tip with bristles 12 proximate the distal delivery opening 14 of the tip, of the type disclosed in U.S. Pat. No. 5,816,804. This tip carries a Luer lock fitting 16 at its proximal end 20 that is adapted to be coupled to the syringe, which carries a mating Luer lock fitting. This first tip can be used to deliver a formulation supragingivally or into the top of gingival, perimplant or periodontal pockets. This can be accomplished by wetting the bristles 12 by delivering a small amount of the formulation out of the tip's opening 14. The wetted bristles can then be moved along and pushed down into the pocket, to deliver the formulation into the pocket. The tip can be made of a material that can be bent and hold a bend, such as an elastomer. This way the tip can be bent to facilitate delivery to teeth, implants or other structures in the mouth. An alternative not shown in the drawing is a tip with a sponge in place of bristles 14. Sponges can be used for shallow periodontal pockets.

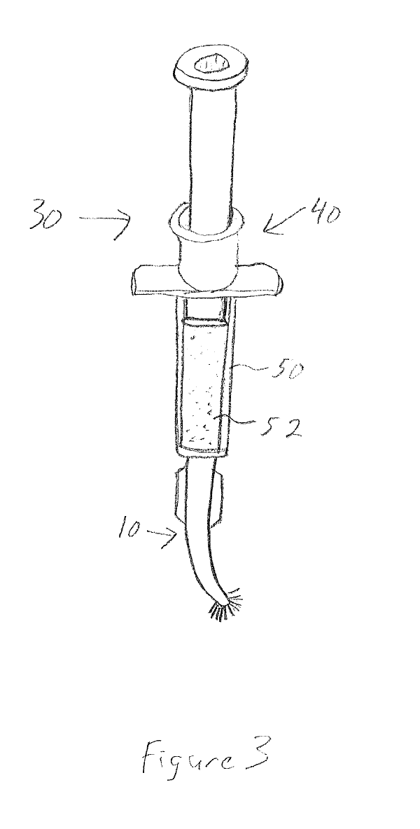

A kit 30 (illustrated assembled and ready to use in FIG. 3) may comprise tip 10 (or a different tip) along with delivery device (e.g., syringe) 40 which has barrel 50 that contains the correct volume of the particular formulation 52 that is to be delivered via tip 10.

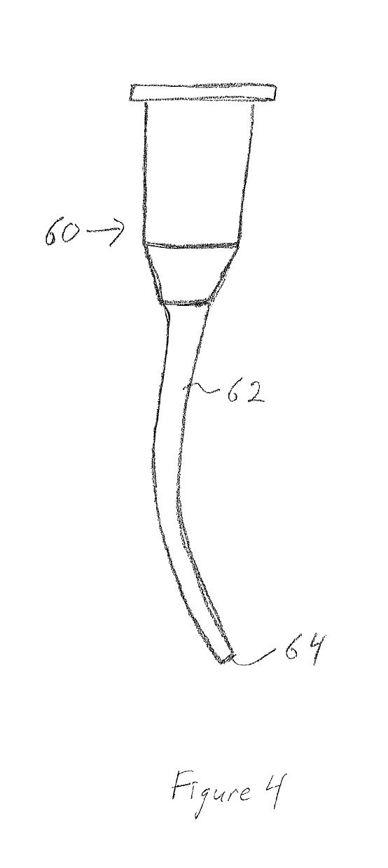

A second tip 60, FIG. 4, has a very thin distal delivery tube 62 that is dimensioned such that it can be inserted into gingival, periodontal or implant pockets. The distal delivery tube may be a straight or curved tube that is 1-10 mm long. One non-limiting example of such a tip can be a microcapillary tip available from Oratech LLC of Riverton, Utah; this tip can be modified (e.g., cut) to change the length of the delivery tube, as desired based on the use of the tip. The tip can be made of a material that can be bent and hold a bend, such as an elastomer. This way the tip can be bent to facilitate delivery to soft tissue, bone, pockets around teeth and implants or other structures in the mouth through outlet orifice 64.

The subject methods for supra and sub-gingival delivery can apply to the above formulations as well as other agents that need to be applied into or within subgingival pockets, or elsewhere in the mouth. There is no limitation as to the specific agent or the treatment regimen accomplished fully or in part with the agent. Other non-limiting types of agents include antimicrobials, fluorides and anti-inflammatories.

The delivery methods contemplate attaching a tip to a pre-filled syringe or other delivery device and then delivering the agent supragingivally, or into a subgingival pocket (or elsewhere in the mouth) via the tip. If the bristled tip is used (the first tip as described above), or a sponge tip, the wetted bristles/sponge can be used to push the agent interproximally, supragingivally, or subgingivally into shallow pockets. For deeper pockets or other indicated areas such as furcations or implant components the non-bristled tip (the second tip as described above, or a similar straight or bent tip) can be used. Both tips can, in one embodiment of the tips, be bent to reach different areas of the mouth. The tips can be moved medially/laterally and/or superiorly/inferiorly during use, as needed. For example, the end of the tip can be placed at or close to the bottom of a pocket and then the tip can be slowly withdrawn from the pocket as an agent is dispensed into the pocket via the tip. This technique can be used so as to fill a pocket with the dispensed agent. Excess agent deposited supragingivally can be rinsed with water and removed by suction. After completion of the procedure the kit is disposable. Either tip can also be used to deliver an agent or combination of agents topically in a supra or sub gingival manner or to other tissues in the mouth.

Delivery methods for the maintenance, surgery, endodontic and comfort (anesthetic) formulations described above are contemplated herein. The specific procedures described herein are illustrative but are not limiting as to the scope of the disclosure herein.

Other features and aspects will be apparent to those skilled in the field from the above description, and are within the scope of the claimed invention.

What is claimed is:

1. A method of delivering a chemical formulation into a mouth of a patient, the method comprising:
delivering the chemical formulation subgingivally, the chemical formulation comprising a tetracycline antibiotic having a pH in the range of about 3.5 to about 9.

2. The method of claim 1, wherein delivering the chemical formulation comprises delivering the chemical formulation from a delivery device via a tip, the tip extending from a proximal end to a distal end, the proximal end configured to be attached to the delivery device and having a proximal opening configured to receive the chemical formulation from the delivery device, and the distal end having a distal opening configured to deliver the chemical formulation into a subgingival part of the mouth of the patient.

3. The method of claim 2, the tip comprising a proximal portion comprising the proximal end and a distal portion comprising the distal end, wherein the distal portion is thinner than the proximal portion.

4. The method of claim 3, wherein the distal portion has a length in the range of 1-10 mm.

5. The method of claim 2, wherein at least a portion of the tip is curved.

6. The method of claim 2, comprising inserting the distal end of the tip into a gingival pocket of the patient and, during delivering the chemical formulation subgingivally, withdrawing the tip from the gingival pocket.

7. The method of claim 1, further comprising providing a syringe prefilled with the chemical formulation.

8. The method of claim 7, further comprising attaching a tip to the syringe, the tip extending from a proximal end to a distal end, the proximal end configured to be attached to the syringe and comprising a proximal opening configured to receive the chemical formulation from the syringe, and the distal end comprising a distal opening configured to deliver the chemical formulation, wherein delivering the chemical formulation subgingivally comprises delivering the chemical formulation from the syringe through the tip into a gingival pocket of the patient.

9. The method of claim 8, further comprising inserting the distal end of the tip into the gingival pocket of the patient and withdrawing the tip from the gingival pocket while delivering the chemical formulation subgingivally.

10. The method of claim 1, wherein the chemical formulation comprises 50 mg/mL tetracycline.

11. The method of claim 1, wherein the chemical formulation comprises 100 mg/mL tetracycline.

12. The method of claim 1, wherein the chemical formulation comprises a liquid, paste, or gel carrier medium.

13. A kit comprising:
a delivery device prefilled with a chemical formulation comprising a tetracycline antibiotic having a pH in the range of about 3.5 to about 9.

14. The kit of claim 13, wherein the delivery device is a syringe.

15. The kit of claim 13, further comprising a tip configured to be coupled to the delivery device and configured to deliver the chemical formulation from the delivery device to a mouth of a patient.

16. The kit of claim 15, wherein the tip is configured to deliver the chemical formulation subgingivally.

17. The kit of claim 15, wherein at least a portion of the tip is curved.

18. The kit of claim 15, wherein the tip extends from a proximal end to a distal end, the proximal end configured to be attached to the delivery device and having a proximal opening configured to receive the chemical formulation from the delivery device, and the distal end having a distal opening configured to deliver the chemical formulation into the mouth of the patient.

19. The kit of claim 13, wherein the delivery device is prefilled with 0.5 mL of the chemical formulation.

20. The kit of claim 13, wherein the delivery device is prefilled with 2 mL of the chemical formulation.
